(19) United States
(12) Patent Application Publication
(10) Pub. No.: US 2016/0310631 A1
(43) Pub. Date: Oct. 27, 2016

(54) ANTIMICROBIAL FIBROUS MATERIALS AND METHODS OF MAKING SAME

(71) Applicant: 3M INNOVATIVE PROPERTIES COMPANY, Saint Paul, MN (US)

(72) Inventors: SHILPI K. SANGHI, SAINT PAUL, MN (US); ALI B. MAHFUZA, MENDOTA HEIGHTS, MN (US)

(73) Assignee: 3M INNOVATIVE PROPERTIES COMPANY, SAINT PAUL, MN (US)

(21) Appl. No.: 15/103,896

(22) PCT Filed: Dec. 9, 2014
(86) PCT No.: PCT/US2014/069198

§ 371 Date: Jun. 13, 2016

Related U.S. Application Data
(60) Provisional application No. 61/918,111, filed on Dec. 19, 2013.

(51) Int. Cl. A61L 15/46; C08B 15/06; A61L 15/44; A61L 15/60; A61L 15/20; A61L 15/28

(52) U.S. Cl. CPC A61L 15/46; A61L 15/20; A61L 15/28; A61L 15/44; A61L 15/60; C08B 15/06; A61L 2300/404; A61L 2300/208; A61L 2300/232

(57) ABSTRACT

Antimicrobial fibrous materials and methods of making same. The antimicrobial fibrous material can include a plurality of absorbent fibers; and a quaternary ammonium compound covalently grafted to the plurality of fibers. The quaternary ammonium compound can be derived from quaternary amine-functionalized ethylenically unsaturated monomers. The method can include providing a plurality of absorbent fibers; irradiating the plurality of fibers with high energy irradiation to generate a plurality of irradiated fibers; providing quaternary amine-functionalized ethylenically unsaturated monomers; and combining the plurality of irradiated fibers with the quaternary amine-functionalized ethylenically unsaturated monomers to form antimicrobial fibers with a quaternary ammonium compound covalently grafted thereto

### FIELD

The present disclosure generally relates to antimicrobial fibrous materials, medical articles comprising antimicrobial fibrous materials, and methods of making antimicrobial fibrous materials.

### BACKGROUND

Healthcare associated infections (HAIs) also known as hospital acquired infections have been increasing and have become a public health concern. Patients with HAIs have longer hospital stays contributing to increased health care costs. Use of preventive interventions can reduce inpatient hospital HAIs by 70%. The pressure on hospitals to reduce HAIs is leading to an increased used of hygiene products and an increasing demand for new antimicrobial solutions.

### SUMMARY

Antimicrobials can kill and/or prevent the growth of bacteria. Antimicrobials can be classified into two types; leachable (e.g., silver (Ag), chlorhexidine gluconate (CHG), and polyhexamethylene biguanide (PHMB)) and non-leachable (e.g., covalently-bound quaternary amines). In some cases, leachable antimicrobials can be associated with concerns of toxicity (e.g., by leaching harmful chemicals into the environment) and/or of generating adaptive organisms. Use of non-leachable antimicrobials can pacify concerns of toxicity and bacterial resistance.

The present disclosure generally relates to antimicrobial fibers and fibrous substrates or materials, and particularly, the present disclosure relates to antimicrobial fibers and fibrous materials comprising non-leachable (i.e., covalently-bound) quaternary ammonium-based compounds. The present disclosure also relates to various articles comprising the antimicrobial fibers or fibrous materials, such as medical articles (e.g., wound dressings; intravenous dressings; packing materials for wounds or surgical incisions; any other any other absorptive packing materials to be used in natural body orifices (e.g., nasal packings); etc.); personal articles (e.g., feminine products, incontinence products, diapers, etc.); cleaning articles (e.g., wipes, cleaning pads, etc.); or combinations thereof. Particularly, the present disclosure refers to absorptive materials or substrates. Absorptive materials can be described as materials that absorb at least 1000 wt % of water, based on the original dry weight of the material; in some embodiments, at least 1500 wt % of water; in some embodiments, at least 2000 wt % of water; and in some embodiments, at least 2400 wt % of water.

Use of the antimicrobial fibers and resulting antimicrobial articles of the present disclosure can suppress growth of bacteria and prevent the spread of bacteria. These antimicrobial fibers and articles of the present disclosure can be used prophylactically, because it is unlikely for pathogens to develop resistance to the quaternary ammonium-based compounds (“quats”). This is at least partially because quats function by binding to bacterial cell membranes and disrupting the barrier function of the membrane, leading to loss of the transmembrane potential, leakage of cytoplasmic contents, and concomitant cell death. The cationic charges in such quats can enhance their affinity to anionic components of the bacterial cell membranes by electrostatic attraction.

Some aspect of the present disclosure provide an antimicrobial fibrous material for use in medical articles. The antimicrobial fibrous material can include a plurality of absorbent fibers; and a quaternary ammonium compound covalently grafted to the plurality of fibers. The quaternary ammonium compound can be derived from quaternary amine-functionalized ethylenically unsaturated monomers, and at least some of the monomers can have the formula of Formula I:

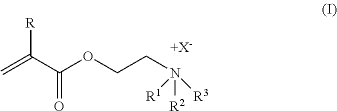

wherein:
R is selected from H and CH₃;
R¹ and R² are each selected from CH₃ and C₂H₅;
R³ is CₙH₂ₙ₊₁, where n ranges from 4 to 22; and
X is selected from Cl, Br, BF₄, N(SO₂CF₃)₂, O₃SCF₃, and O₃SC₄F₉.

Some aspects of the present disclosure provide a method of making antimicrobial fibers for use in medical articles. The method can include providing a plurality of absorbent fibers; irradiating the plurality of fibers with high energy irradiation to generate a plurality of irradiated fibers; and providing quaternary amine-functionalized ethylenically unsaturated monomers. At least some of the monomers can have the formula of Formula I above. The method can further include combining the plurality of irradiated fibers with the quaternary amine-functionalized ethylenically unsaturated monomers to form antimicrobial fibers with a quaternary ammonium compound covalently grafted thereto.

Other features and aspects of the present disclosure will become apparent by consideration of the detailed description and accompanying drawings.

### BRIEF DESCRIPTION OF THE DRAWINGS

FIG. 1 is a schematic perspective view of an article comprising an antimicrobial fibrous material according to one embodiment of the present disclosure.

FIG. 2 is a schematic illustration of a method of making antimicrobial fibers according to one embodiment of the present disclosure.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “affixed,” and “coupled” and variations thereof are used broadly and encompass both direct and indirect affixations and couplings. It is to be understood that other embodiments may be utilized, and structural or logical changes may be made without departing from the scope of the present disclosure.

The present disclosure generally relates to antimicrobial fibers and fibrous substrates, and methods for making same. The present disclosure further relates to articles, particularly medical articles, comprising the antimicrobial fibers and/or fibrous substrates. Particularly, the present disclosure relates to antimicrobial fibers that are formed by irradiation-grafting the fibers with quaternary ammonium-based compounds, without the use of catalysts, to form antimicrobial fibers comprising non-leachable quaternary ammonium-based compounds grafted (i.e., irradiated-grafted) thereto. For example, the quaternary ammonium-based compounds can be grafted to an outer surface of the fibers, such that the quaternary ammonium-based compounds extend from the outer surfaces of the fibers (e.g., in a brush-like configuration). That is, according to the present disclosure, irradiation grafting can be used to grow quaternary ammonium-based “brushes” (e.g., “quaternary ammonium polymer brushes”) on activated fibers (e.g., activated cellulose fibers). As exemplified in the Examples section, the present inventors found that non-leachable quaternary ammonium compounds covalently attached to fibers, according to the present disclosure, can kill more than 99.99% of most common gram negative and gram positive bacteria. The antimicrobial fibers can be converted into articles (particularly, medical articles) that can act as a barrier, for example, between a wound and environmentally-present pathogens.

Generally, “non-leaching” or “non-leachable” antimicrobials refers to antimicrobial materials that are covalently bonded to the chemical structure making up the fibers. Particularly, “non-leaching” means that the quaternary ammonium compounds of the present disclosure do not separate from the fiber or otherwise become non-integral with the fiber under standard uses or exposure conditions.

The antimicrobial-functionalized fibers and articles of the present disclosure are generally initiated from free radicals formed on the fibers by ionizing radiation and the subsequent graft-polymerization of quaternary ammonium-based monomers in an aqueous solution. Specifically, fibers of the present disclosure can be grafted with quaternary ammonium compounds of the present disclosure by irradiating them to form free radicals. The choice of fiber depends upon the final application. The nature of the fiber dictates the amount of free radicals formed per unit fiber when exposed to high energy radiation. This in turn controls the average molecular weight of quaternary ammonium compound (e.g., polymer) grafted onto the fiber, and the density of the polymer brushes on the fibers, which controls the processability of the fibers and the antimicrobial efficacy.

The antimicrobial materials of the present disclosure can be applied to the fibers after formation of the fibers, while still allowing for the antimicrobial material to be covalently bonded to the fiber. That is, in methods of the present disclosure, the antimicrobial material is applied to the fiber after formation of the fiber, rather than during formation of the fiber.

As a result, irradiated (i.e., activated) fibers employed in the antimicrobial fibers of the present disclosure can be combined with the antimicrobial material such that the antimicrobial material covalently bonds with the fibers, e.g., forming carbon-carbon bonds between the chemical structure of the fiber and the antimicrobial material. For example, the antimicrobial material can include one or more moieties configured to chemically react (i.e., to form a covalent bond) with free radicals of the activated fibers, i.e., after the fibers have been irradiated with high energy irradiation. Such free radicals may be present on an outer surface of the fiber. As a result, the antimicrobial material can covalently bond with the chemical structure of the fiber via non-siloxane covalent bonds.

Antimicrobial materials of the present disclosure can include quaternary ammonium compounds. Quaternary ammonium compounds generally act by disrupting a cell membrane. Quaternary ammonium compounds, and particularly, quaternary ammonium compounds comprising an alkyl chain, can bind by ionic and hydrophobic interactions to the surface of microbial membranes, such that the cationic head is facing outwards and the hydrophobic tail is inserted into the lipid bilayer of the microbial membrane. This can cause membrane damage and leakage of intracellular constituents from the cell, ultimately resulting in cell death.

The antimicrobial material can covalently attach to the fiber, and in some embodiments, can further bond to other adjacent antimicrobial materials (i.e., quaternary ammonium compounds) to form crosslinks with other quaternary ammonium compounds that may, in turn, be covalently bonded to the fiber.

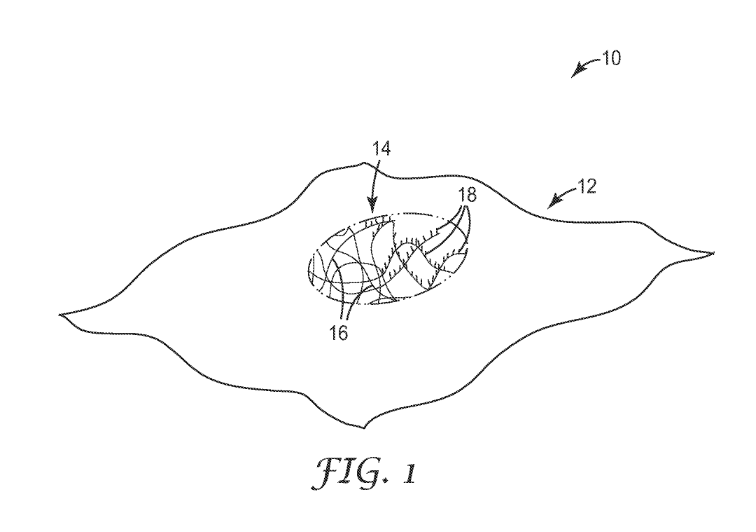

FIG. 1 is a schematic illustration of an article (e.g., a medical article) 10 according to one embodiment of the present disclosure. The article 10 includes an antimicrobial fibrous material or substrate 12 according to one embodiment of the present disclosure. The antimicrobial fibrous material 12 comprises antimicrobial fibers 14, which comprise a plurality of fibers 16 covalently grafted with an antimicrobial, i.e., a quaternary ammonium compound (“quat”) 18. Particularly, the grafted quat can include monomers (i.e., single-unit quats grafted to the fiber), oligomers, polymers, and combinations thereof. That is, monomeric quaternary ammonium compounds of the present disclosure can polymerize simultaneously with grafting to the fiber to form a combination of grafted monomers, oligomers and polymers. In many cases, following the methods of the present disclosure, the grafted quat will be in the form of oligomers and polymers.

In some embodiments, the term “oligomer” can refer to a molecular chain of 2 to 50 repeat units, in some embodiments, 2 to 40 units, in some embodiments, 5 to 30 units, in some embodiments, 10 to 30 units, and in some embodiments, 20 to 30 units.

In some embodiments, the term “polymer” can refer to a molecular chain of at least 50 repeat units, in some embodiments, at least 100 repeat units, in some embodiments, at least 500 repeat units, in some embodiments, at least 1000 repeat units, in some embodiments, at least 5000 units, and in some embodiments, at least 10,000 units.

As described above, in some embodiments, the antimicrobial material can include a quaternary ammonium compound, which can include non-silane-based quaternary ammonium compounds. In some embodiments, such a quaternary ammonium compound can include a polyacrylate quaternary ammonium oligomer, polymer, or a combination thereof. In some embodiments, polymerization of the quaternary ammonium compound (i.e., to form a polyacrylate), as well as reaction of the quaternary ammonium compound with the fiber, occur simultaneously.

In some embodiments, the quaternary ammonium compound 18 can be derived from quaternary amine-functionalized (or quaternary ammonium) ethylenically unsaturated monomers. That is, in some embodiments, the quaternary ammonium compound can include one or more monomers having the structural formula of Formula I:

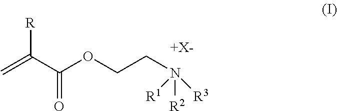

where:

R is selected from H and CH₃;

R¹ and R² are each selected from CH₃ and C₂H₅;

R³ is CₙH₂ₙ₊₁, where n ranges from 4 to 22; and

X is selected from Cl, Br, BF₄, N(SO₂CF₃)₂, O₃SCF₃, and O₃SC₄F₉.

In some embodiments, R³ can be selected from selected from C₄H₉, C₆H₁₃, C₁₀H₂₁, C₁₂H₂₅, C₁₆H₃₃, C₁₈H₃₇, C₂₀H₄₁, and C₂₂H₄₅. In some embodiments, particularly suitable values for “n” can range from 4 to 16, in some embodiments, from 6 to 16, in some embodiments, from 10 to 16, and in some embodiments, from 10 to 12.

Suitable examples of quaternary amine-functionalized ethylenically unsaturated monomers can include dimethyldecylammoniumethylacrylate (DMAEA-C₁₀), dimethyldecylammoniumethylmethacrylate (DMAEMA-C₁₀), dimethylhexadecylammoniumethylacrylate (DMAEA-C₁₆), and dimethylhexadecylammoniumethylmethacrylate (DMAEMA-C₁₆).

Specific examples of suitable quaternary amine-functionalized ethylenically unsaturated monomers can include dimethylhexadecylammoniumethylacrylate halides (DMAEA-C₁₆ halides; e.g., dimethylhexadecylammoniumethylacrylate bromides (DMAEA-C₁₆Br)); dimethyldecylammoniumethylacrylate halides (DMAEA-C₁₀ halides; e.g., dimethyldecylammoniumethylacrylate bromides (DMAEA-C₁₀Br)), dimethyl-hexadecylammoniumethylmethacrylate halides (DMAEMA-C₁₆ halides; e.g., dimethyl-hexadecylammoniumethylmethacrylate bromides (DMAEMA-C₁₆Br)); dimethyl-decylammoniumethylmethacrylate halides (DMAEMA-C₁₀ halides; e.g., dimethyl-decylammoniumethylmethacrylate bromides (DMAEMA-C₁₀Br)) and derivatives thereof.

Examples of suitable DMAEA-Cₙ halides and DMAEMA-Cₙ halides include derivatives of DMAEA-C₁₆Br and DMAEMA-C₁₆Br, as described below, but it should be understood that similar derivatives of other DMAEA-Cₙ halides and DMAEMA-Cₙ halides are within the spirit and scope of the present disclosure, and one of ordinary skill in the art would understand how to extend the description below regarding the formation of DMAEA-C₁₆Br and DMAEMA-C₁₆Br to such other halides.

Suitable DMAEMA derivatives (e.g., DMAEMA-CₙBr) have the structural formula of Formula II:

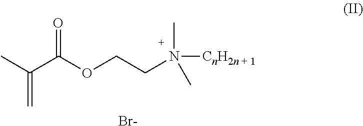

where suitable values for “n” range from 4-22, with particularly suitable values for “n” ranging from 4-16, in some embodiments, from 6-16, in some embodiments, from 10-16, and in some embodiments, from 10-12. Such polymer-chain lengths allow the DMAEMA derivative to move enough within the cross-linked matrix while also preventing the DMAEMA derivative from phase separating from the resulting cross-linked matrix.

By way of example only, DMAEMA-C₁₆Br and its derivatives may be formed by combining dimethylaminoethylmethacrylate salt, acetone, 1-bromohexadecane, and optionally, an antioxidant. The mixture may be stirred for about 16 hours at about 35° C. and then allowed to cool to room temperature. The resulting white solid precipitate may then be isolated by filtration, washed with cold ethyl acetate, and dried under vacuum at 40° C.

Similarly, DMAEA-C₁₆Br and its derivatives may be formed by combining dimethylaminoethylacrylate, of acetone, 1-bromohexadecane, and optionally, an antioxidant. The mixture may be stirred for 24 hours at 35° C., and then allowed to cool to room temperature. The acetone may then be removed by rotary evaporation under vacuum at 40° C. The resulting solids may then be washed with cold ethyl acetate and dried under vacuum at 40° C.

Other DMAEMA-CₙBr or DMAEA-CₙBr based monomers were made following the same procedure outlined above where n varied between 4 and 22. Other DMAEMA-CₙX based monomers can be made using an ion exchange reaction. Several of those monomers were used in the Examples described below.

The quaternary ammonium compound 18 grafted to the fibers to form the antimicrobial fibers 14 can include a variety of different quaternary ammonium compounds derived from a variety of different monomers. That is, a variety of monomers can be combined with a plurality of irradiated fibers to form the antimicrobial fibers 14 comprising a variety of monomers, oligomers and/or polymers of different combinations of various monomers, such that the resulting antimicrobial fibers 14 include a random distribution and arrangement of various quaternary ammonium compounds.

The alkyl chain length (i.e., n of Formulas I and II) can cause a change in both conformation and charge density of the grafted antimicrobial, which in turn can influence the mode of interaction with a cytoplasmic membrane. For the quats of the present disclosure, a hydrophilic-lipophilic balance can influence the antimicrobial properties. As shown in the Examples section, the antimicrobial activity of quats can increase with increasing alkyl chain length (i.e., increasing hydrophobicity), in some cases, up to about n=10 (i.e., C₁₀H₂₁), after which a decrease in antimicrobial activity can occur with an increase in chain length (i.e., decreasing hydrophilicity). Also, excessive hydrophobicity can give rise to nonspecific binding, which is some cases may result in lysis of human red blood cells.

The unreacted fibers (i.e., starting material) of the present disclosure can already be in the form of a woven, a nonwoven, or a combination thereof when the fibers are reacted (i.e., covalently grafted) with the antimicrobial material. Alternatively, in some embodiments, the fibers can be formed into a woven, a nonwoven, or a combination thereof, after being reacted (i.e., covalently grafted) with the antimicrobial material.

A woven substrate or fabric is generally formed by weaving and typically only stretches diagonally on the bias directions (between the warp and weft directions), unless the fibers forming the woven are elastic.

A nonwoven substrate is a nonwoven web which may include nonwoven webs manufactured by any of the commonly known processes for producing nonwoven webs. As used herein, the term “nonwoven web” refers to a fabric that has a structure of individual fibers or filaments which are randomly and/or unidirectionally interlaid in a mat-like fashion.

For example, a fibrous nonwoven web can be made by carded, air laid, wet laid, spunlaced, spunbonding, electrospinning or melt-blowing techniques, such as melt-spun or melt-blown, or combinations thereof. Spunbonded fibers are typically small diameter fibers that are formed by extruding molten thermoplastic polymer as filaments from a plurality of fine, usually circular capillaries of a spinneret with the diameter of the extruded fibers being rapidly reduced. Meltblown fibers are typically formed by extruding the molten thermoplastic material through a plurality of fine, usually circular, die capillaries as molten threads or filaments into a high velocity, usually heated gas (e.g. air) stream which attenuates the filaments of molten thermoplastic material to reduce their diameter. Thereafter, the meltblown fibers are carried by the high velocity gas stream and are deposited on a collecting surface to from a web of randomly disbursed meltblown fibers. Any of the non-woven webs may be made from a single type of fiber or two or more fibers that differ in the type of thermoplastic polymer and/or thickness.

Staple fibers may also be present in the fibrous substrates, materials or webs of the present disclosure. The presence of staple fibers can provide a loftier, less dense web than a web of only melt blown microfibers.

The fibers of the nonwoven substrate typically have an effective fiber diameter of from about 3 to 20 micrometers preferably from about 4 to 10 micrometers, as calculated according to the method set forth in Davies, C. N., “The Separation of Airborne Dust and Particles,” Institution of Mechanical Engineers, London, Proceedings 1B, 1952.

Absorbent fibers are of particular interest in forming medical articles, such as wound dressings, since absorbent fibers can absorb excess liquid in a reasonable amount of time and can release liquid into the environment in an equally reasonable amount of time. Specifically, as used herein, “absorbent fibers” are fibers that absorb at least 1000 wt % of water, based on the original dry weight of the fibers. In some embodiments, “absorbent fibers” are fibers that absorb at least 1500 wt % of water, based on the original dry weight of the fibers; in some embodiments, at least 2000 wt %; and in some embodiments, at least 2400 wt %.

Examples of absorbent fibers can include, but are not limited to, cellulose fibers (e.g., Lyocell TENCEL® fibers of 1.7 dtex*38 mm (available from Lenzing); rayon fibers, treated cellulose fibers (e.g., carboxymethylated cellulose fibers), other suitable cellulose fibers, or a combination thereof); other suitable absorbent fibers; or a combination thereof.

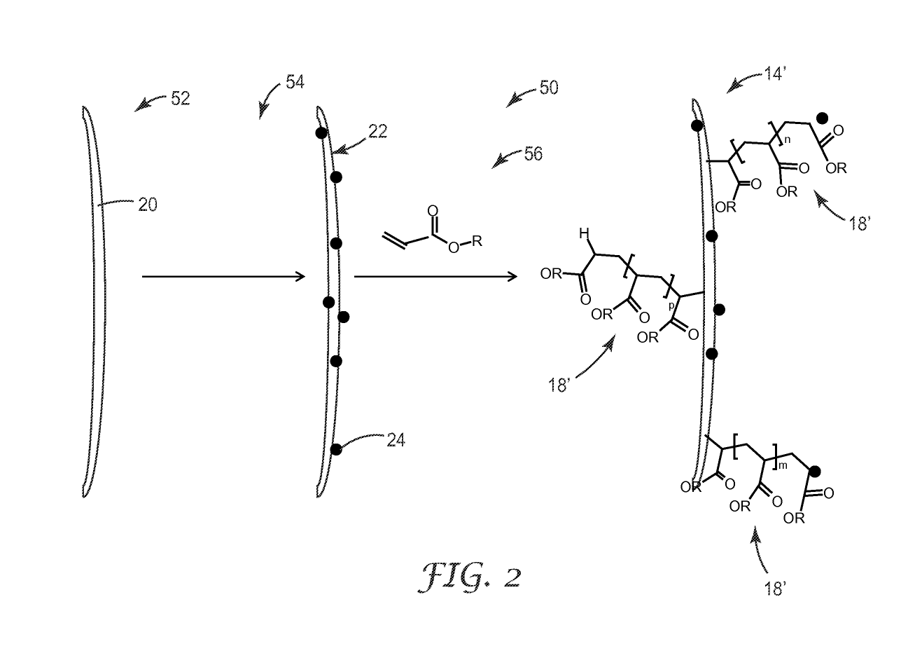

The antimicrobial fibrous material 12, and accordingly, the article 10 can be catalyst-free, which can be a direct result of the method of making the antimicrobial fibrous material 12, which is described in greater detail below with respect to FIG. 2. Catalysts or initators can be toxic and/or hazardous and can leach out of the resulting antimicrobial fibrous material 12, if present, e.g., into a wound if the article 10 is a wound dressing. Such leaching of toxic materials is particularly disadvantageous in the field of medical articles. In the present disclosure, “catalyst-free” or “free of catalyst” can refer to a material or article that is 100% free of catalysts or initators, or at least includes no catalysts or initators that were intentionally added during processing or otherwise.

Examples of such catalysts or initiators can include, but are not limited to, organic metal compounds such as compounds comprising cerium (IV), copper, tin, or a combination thereof.

The antimicrobial fibers 14 (and accordingly, the antimicrobial fibrous material 12 and the article 10) are particularly suited to reduce microbial growth, e.g., microbes that can cause malodor. As a result, the antimicrobial fibers 14 of the present disclosure include sufficient antimicrobial material to substantially inhibit microbial growth. In some embodiments, substantially reducing microbial growth can include exhibiting at least a 1 log reduction, in some embodiments, at least a 2 log reduction, in some embodiments, at least a 3 log reduction, in some embodiments, at least a 5 log reduction, and in some embodiments, at least a 6 log reduction in either gram positive or gram negative bacteria, e.g., when tested pursuant to the Microbial Kill test method set forth in the Examples.

In some embodiments, sufficient antimicrobial material in the resulting antimicrobial fibers 14 can include at least (or more than) 0.1 parts by weight of quaternary ammonium compound per 100 parts by dry weight of the fibers (0.1 wt %); in some embodiments, at least (or more than) 0.25 wt % of quaternary ammonium compound; in some embodiments, at least (or more than) 0.5 wt % of quaternary ammonium compound; in some embodiments, at least (or more than) 1 wt % of quaternary ammonium compound; in some embodiments, at least (or more than) 2 wt % of quaternary ammonium compound; in some embodiments, at least (or more than) 5 wt % of quaternary ammonium compound; in some embodiments, at least (or more than) 10 wt % of quaternary ammonium compound; in some embodiments, at least (or more than) 20 wt % of quaternary ammonium compound; and in some embodiments, at least (or more than) 25 wt % of quaternary ammonium compound.

In some embodiments, sufficient antimicrobial material in the resulting antimicrobial fibers 14 can include from 0.1 wt % to 25 wt % of quaternary ammonium compound; and in some embodiments, from 2 wt % to 5 wt % of quaternary ammonium compound.

FIG. 2 schematically illustrates a method 50 of making antimicrobial fibers according to one embodiment of the present disclosure. Generally, in some embodiments, the process of preparing the antimicrobial fibers of the present disclosure comprises the steps of providing a plurality of fibers (i.e., absorbent fibers), exposing the fibers to high energy radiation in an inert atmosphere, and subsequently imbibing the activated (i.e., irradiated) fibers with a solution or suspension comprising the grafting monomers to graft-polymerize said monomers to the surface of the fibers.

As shown in FIG. 2, in a first step 52, a plurality of fibers 20 can be provided (one representative fiber 20 is shown for simplicity). The plurality of fibers 20 can be purged of oxygen in an inert atmosphere. Generally, the fibers are placed in a chamber purged of oxygen. Typically, the chamber comprises an inert atmosphere such as nitrogen, carbon dioxide, helium, argon, etc. with a minimal amount of oxygen (less than 100 ppm). An atmosphere of at least 100 ppm of oxygen is generally known to inhibit free-radical polymerization. Desirably, the surface of the imbibing vessel is inert to the ionizing radiation.

As shown in a second step 54, the plurality of fibers 20 can be irradiated with a high energy radiation (i.e., in the inert atmosphere), such as electron beam (e-beam) radiation and/or gamma radiation, to generate a plurality of irradiated fibers 22 comprising free radicals 24. In a third step 56, quaternary amine-functionalized ethylenically unsaturated monomers, represented by Formula III, are provided:

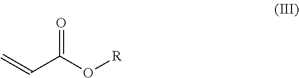

which is a shorthand or schematic representation of any of the quaternary amine-functionalized ethylenically unsaturated monomers described above, where R includes the quaternary ammonium. Particularly, Formula III can represent any of the DMAEA-CₙX or DMAEMA-CₙX monomers described above. Formula III can also represent a mixture of a variety of such monomers.

The monomers can then be combined with the plurality of irradiated fibers 22 (e.g., the irradiated fibers 22 can be imbibed with an aqueous solution of the monomers) to form antimicrobial fibers 14′ with quaternary ammonium compounds 18′ covalently grafted thereto, via non-siloxane covalent bonds (e.g., via carbon-carbon bonds, as shown). The reaction can occur in an inert atmosphere (e.g., the same inert atmosphere in which the plurality of fibers 20 were irradiated) and can be quenched by exposing the reaction solution to atmospheric oxygen. The antimicrobial fibers 14′ can further include residual monomers that can optionally be washed from the antimicrobial fibers 14′ and/or residual free radicals that can be quenched.

By way of example only, the three quaternary ammonium compounds 18′ shown in FIG. 2 are each illustrated as being oligomeric or polymeric in nature but can be of varying lengths. That is, “n,” “p” and “m” can have different values. In addition, the various combinations of monomers, or monomeric units, making up each of the illustrated oligomers/polymers can be different. As described above, polymerization of the monomers can occur simultaneously with grafting of the monomers to the irradiated fibers 22. Two of the three illustrated quaternary ammonium compounds 18′ are also shown by way of example as including a free radical to illustrate that these compounds may still be undergoing polymerization. Available free radicals on the quaternary ammonium compounds 18′ can be used to add additional units to the same oligomer or polymer, or the free radicals can cause one grafted quaternary ammonium compound to covalently bind to an adjacent grafted quaternary ammonium compound, thereby forming cross-links amongst the quaternary ammonium compounds 18′.

In some embodiments, the monomers may be grafted onto the fibers in a single reaction step (i.e., exposure to an ionizing radiation) followed by imbibing with all grafting monomers present; or in sequential reaction steps (i.e., a first exposure to ionizing radiation followed by imbibing with one or more grafting monomer, then a second exposure to an ionizing radiation and a second imbibing after the second exposure to the ionizing radiation).

It will be further understood that the grafting process will yield a radical species on the surface of the fibers. After imbibing with the monomer solution, polymerization will initiate with the formation of a radical on the monomer that may further polymerize with one or more additional monomers, resulting in grafted polymers having the quaternary ammonium compounds pendent from the polymer chain.

The irradiation step comprises the high energy, or ionizing, irradiation of the fibers, preferably with ionizing e-beam or gamma radiation to prepare free radical reaction sites on surfaces of such fibers, upon which the monomers are subsequently grafted.

The phrase “high energy radiation” or “ionizing irradiation” refers to radiation of a sufficient dosage and energy to cause the formation of free radical reaction sites on the surface(s) of the base substrate. High energy radiation may include gamma, electron-beam, x-ray and other forms of electromagnetic radiation. In some instances, corona radiation can be sufficiently high energy radiation. The radiation is sufficiently high energy that when absorbed by the surfaces of the fibers, sufficient energy is transferred to the fibers to result in the cleavage of chemical bonds in the fibers and the resultant formation of free radical sites on the fibers.

High energy radiation dosages are measured in kilograys (kGy), or rads. Doses can be administered in a single dose of the desired level or in multiple doses which accumulate to the desired level. Dosages can range cumulatively from about 1 kGy (0.1 MRad) to about 200 kGy (20 MRad). The dose can be delivered all at once such as from an E-beam source or accumulated from a slow dose rate over several hours such as dosage delivered from a gamma source. The total dose received by the substrate depends on a number of parameters including source activity, residence time (i.e. the total time the sample is irradiated), the distance from the source, and attenuation by the intervening cross-section of materials between the source and sample. Dose is typically regulated by controlling residence time, distance to the source, or both.

Generally, it was found that doses in the range of about 40 to 70 kGy (4-7 MRad) were suitable for generating the antimicrobial fibers of the present disclosure. Total dose requirement for any given composition will vary as a function of desired grafting objectives, monomer selected, fiber used and the dose rate. Thus, a dose rate can be selected based on desired properties for a specified composition. The dose rate is typically in the range of 0.0005 kGy/sec (50 rad/sec), i.e., for gamma radiation, to 200 kGy/sec (20 MRad/sec), i.e., for E-beam radiation.

Electron beam is one preferred method of grafting due to the ready-availability of commercial sources. Electron beam generators are commercially available from a variety of sources, including the ESI “ELECTROCURE” EB SYSTEM from Energy Sciences, Inc. (Wilmington, Mass.), and the BROADBEAM EB PROCESSOR from PCT Engineered Systems, LLC (Davenport, Iowa). For any given piece of equipment and irradiation sample location, the dosage delivered can be measured in accordance with ASTM/ISO 5127S entitled “Practice for Use of a Radiochromic Film Dosimetry System.” By altering extractor grid voltage, and/or distance to the source, various dose requirements can be obtained.

Other sources of irradiation may be used with equal grafting performance; a desirable source of ionizing radiation comprises an electron beam source because the electron beam can produce high and fast dose delivery rates. Electron beams (e-beams) are generally produced by applying high voltage to tungsten wire filaments retained between a repeller plate and an extractor grid within a vacuum chamber maintained at about 10⁻⁶ Torr. The filaments are heated at high current to produce electrons. The electrons are guided and accelerated by the repeller plate and extractor grid towards a thin window of metal foil. The accelerated electrons, traveling at speeds in excess of 10⁷ meters/second (m/sec) and possessing about 100 to 300 kilo-electron volts (keV), pass out of the vacuum chamber through the foil window and penetrate whatever material is positioned immediately beyond the foil window.

The quantity of electrons generated is directly related to the current. As extractor grid voltage is increased, the acceleration or speed of electrons drawn from the tungsten wire filaments increases. E-beam processing can be extremely precise when under computer control, such that an exact dose and dose rate of electrons can be directed against the fibers.

The temperature within the chamber is desirably maintained at an ambient temperature by conventional means. Without intending to be limited to any particular mechanism, it is believed that the exposure of the fibers to an electron beam results in free radical sites on the fiber surface which can then subsequently react with the grafting monomers in the imbibing or combining step.

The total dose received by the fibers primarily affects the number of radical sites formed on the surface thereof and subsequently the extent to which the grafting monomers are grafted onto the fibers. Dose is dependent upon a number of processing parameters, including voltage, web- or line-speed and beam current. Dose can be conveniently regulated by controlling line speed (i.e., the speed with which the fibers pass under the irradiation device), and the current supplied to the extractor grid. A target dose (e.g., <10 kGy, or <1 MRad) can be conveniently calculated by multiplying an experimentally measured coefficient (a machine constant) by the beam current and dividing by the web speed to determine the exposure. The machine constant varies as a function of beam voltage.

While the controlled amount of electron beam radiation exposure is dependent upon the residence time, the fibers are subjected to a controlled amount of dosage ranging from a minimum dosage of about 1 kGy (0.1 MRad) to a practical maximum dosage of less than about 200 kGy (20 MRad), depending on the particular desired quaternary ammonium compound. Generally, suitable gamma ray sources emit gamma rays having energies of 400 keV or greater. Typically, suitable gamma ray sources emit gamma rays having energies in the range of 500 keV to 5 MeV. Examples of suitable gamma ray sources include cobalt-60 isotope (which emits photons with energies of approximately 1.17 and 1.33 MeV in nearly equal proportions) and cesium-137 isotope (which emits photons with energies of approximately 0.662 MeV). The distance from the source can be fixed or made variable by changing the position of the target or the source. The flux of gamma rays emitted from the source generally decays with the square of the distance from the source and duration of time as governed by the half-life of the isotope.

In some embodiments of the present disclosure, the irradiated fibers, having free radical sites on the surfaces thereof, are imbibed with the aqueous monomer solution or suspension subsequent to and not concurrent with, the irradiation step. The free radical sites generated on the surface of the fibers have average lifetimes ranging from several minutes to several hours and progressively decay to a low concentration within about ten hours at room temperature. Lower temperatures, such as dry ice temperatures, promote longer radical lifetimes. The effective binding absorption capacity of the grafted fibers from the graft polymerization process is little changed after a reaction time of about 12 hours, kept under inert conditions.

Generally, the irradiated fibers are imbibed with the monomer solution immediately after the irradiation step. Generally, when using e-beam, the irradiated fibers are imbibed within an hour, preferably within ten minutes. Generally, when using gamma as a source, the fibers are imbibed immediately after irradiation since irradiation residence time will be relatively long. It has been observed that if the fibers are irradiated by high energy radiation in the presence of the grafting monomers, the grafting yield may be lower.

In the imbibing step, the irradiated fibers are combined, i.e., contacted, with one or more grafting monomers (e.g., in an imbibing solution or suspension containing the monomer(s)). Suitable methods of imbibing include, but are not limited to, a spray coating, flood coating, knife coating, Meyer bar coating, dip coating, and gravure coating.

The imbibing solution remains in contact with the fibers for a time sufficient for the radical sites to initiate polymerization with the grafting monomers. When imbibed with a solution of monomers, grafting reactions are mostly completed after 12 hours exposure; generally about 90+ percent. As a result, the fibers comprise grafted quats attached to the outer surfaces of the fibers.

The concentration of each grafting monomer in the imbibing solution may vary depending on a number of factors including, but not limited to, the nature of grafting monomer or monomers in the aqueous imbibing solution or suspension, the extent of grafting desired, the reactivity of the grafting monomer(s), and the solubility of the monomers used. Typically, the total concentration of the monomers in the imbibing solution ranges from about 1 wt % to about 50 wt %, desirably, from about 5 wt % to about 40 wt %, and more desirably from about 15 wt % to about 30 wt % based on a total weight of the imbibing solution. In some embodiments, the weight of the grafting monomers of the imbibing solution is 0.5 to 5, preferably 1 to 3, times the weight of the fibers.

As mentioned above, once the fibers have been imbibed for a desired period of time, the resulting antimicrobial fibers may be optionally rinsed to remove residual monomer.

In the optional rinsing step, the antimicrobial fibers are washed or rinsed one or more times to remove any unreacted monomers, solvent or other reaction by-products. Typically, the antimicrobial fibers are washed or rinsed up to three times using a water rinse, an alcohol rinse, a combination of water and alcohol rinses, and/or a solvent rinse (e.g., acetone, methyl ethyl ketone, etc). When an alcohol rinse is used, the rinse may include one or more alcohols including, but not limited to, isopropanol, methanol, ethanol, or any other alcohol that is practical to use and an effective solvent for any residual monomer. In each rinse step, the antimicrobial fibers may pass through a rinse bath or a rinse spray.

In the optional drying step, the antimicrobial fibers are dried to remove any rinse solution. Typically, the antimicrobial fibers are dried in oven having a relatively low oven temperature for a desired period of time (referred to herein as “oven dwell time”). Oven temperatures typically range from about 30° C. to about 120° C., while oven dwell times typically range from about 120 to about 600 seconds. Any conventional oven may be used in the optional drying step. The resulting antimicrobial fibers generally include polymer tendrils that are initiated from, and supported by, the fibers.

The embodiments described above and illustrated in the figures are presented by way of example only and are not intended as a limitation upon the concepts and principles of the present disclosure.

All references and publications cited herein are expressly incorporated herein by reference in their entirety into this disclosure.

The following embodiments are intended to be illustrative of the present disclosure and not limiting.

### Embodiments

Embodiment 1 is an antimicrobial fibrous material for use in medical articles comprising:

a plurality of absorbent fibers; and

a quaternary ammonium compound covalently grafted to the plurality of fibers, wherein the quaternary ammonium compound is derived from quaternary amine-functionalized ethylenically unsaturated monomers, and wherein at least some of the monomers have the formula of Formula I:

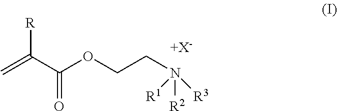

wherein:
R is selected from H and CH₃;
R¹ and R² are each selected from CH₃ and C₂H₅;
R³ is CₙH₂ₙ₊₁, where n ranges from 4 to 22; and
X is selected from Cl, Br, BF₄, N(SO₂CF₃)₂, O₃SCF₃, and O₃SC₄F₉.

Embodiment 2 is the antimicrobial fibrous material of embodiment 1, wherein the antimicrobial fibrous material is free of catalyst.

Embodiment 3 is the antimicrobial fibrous material of embodiment 1 or 2, wherein the antimicrobial fibrous material forms at least a portion of at least one of a wound dressing, an intravenous dressing, a wound or surgical packing material, a bodily orifice packing material, and a combination thereof.

Embodiment 4 is the antimicrobial fibrous material of any of embodiments 1-3, wherein the antimicrobial fibrous material exhibits at least a 2 log reduction in gram positive or gram negative bacteria.

Embodiment 5 is a method of making antimicrobial fibers for use in medical articles, the method comprising:

providing a plurality of absorbent fibers;

irradiating the plurality of fibers with high energy irradiation to generate a plurality of irradiated fibers;

providing quaternary amine-functionalized ethylenically unsaturated monomers, wherein at least some of the monomers have the formula of Formula I:

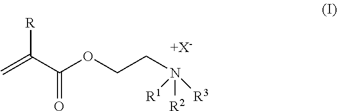

wherein:
R is selected from H and CH₃;
R¹ and R² are each selected from CH₃ and C₂H₅;
R³ is CₙH₂ₙ₊₁, where n ranges from 4 to 22; and
X is selected from Cl, Br, BF₄, N(SO₂CF₃)₂, O₃SCF₃, and O₃SC₄F₉; and

combining the plurality of irradiated fibers with the quaternary amine-functionalized ethylenically unsaturated monomers to form antimicrobial fibers with a quaternary ammonium compound covalently grafted thereto.

Embodiment 6 is the method of embodiment 5, wherein combining the plurality of irradiated fibers with the quaternary amine-functionalized ethylenically unsaturated monomers occurs without the use of catalyst.

Embodiment 7 is the method of embodiment 5 or 6, wherein the antimicrobial fibers are catalyst-free.

Embodiment 8 is the method of any of embodiments 5-7, further comprising removing residual monomers from the antimicrobial fibers.

Embodiment 9 is the method of embodiment 8, wherein removing residual monomers from the plurality of irradiated fibers includes washing the fibers with water.

Embodiment 10 is the method of embodiment 8 or 9, further comprising drying the antimicrobial fibers after removing residual monomers from the antimicrobial fibers.

Embodiment 11 is the method of any of embodiments 5-10, wherein providing quaternary amine-functionalized ethylenically unsaturated monomers includes providing an aqueous solution of quaternary amine-functionalized ethylenically unsaturated monomers, and wherein combining the irradiated fibers with the quaternary amine-functionalized ethylenically unsaturated monomers includes positioning the irradiated fibers in the aqueous solution of quaternary amine-functionalized ethylenically unsaturated monomers.

Embodiment 12 is the method of any of embodiments 5-11, wherein the high energy irradiation includes at least one of electron beam irradiation and gamma irradiation.

Embodiment 13 is the method of any of embodiments 5-12, wherein irradiating the plurality of fibers occurs in an atmosphere comprising less than 20 ppm of oxygen.

Embodiment 14 is the method of any of embodiments 5-13, wherein irradiating the plurality of fibers with a high energy irradiation and combining the plurality of irradiated fibers with the quaternary amine-functionalized ethylenically unsaturated monomers occur in an inert atmosphere.

Embodiment 15 is the method of any of embodiments 5-14, wherein combining the plurality of irradiated fibers with the quaternary amine-functionalized ethylenically unsaturated monomers occurs in an atmosphere comprising less than 20 ppm of oxygen.

Embodiment 16 is the method of any of embodiments 5-15, wherein combining the plurality of irradiated fibers with the quaternary amine-functionalized ethylenically unsaturated monomers initiates a polymerization reaction, and wherein the polymerization reaction is quenched with atmospheric oxygen.

Embodiment 17 is the method of any of embodiments 5-16, wherein combining the plurality of irradiated fibers with the quaternary amine-functionalized ethylenically unsaturated monomers yields antimicrobial fibers comprising at least 2 wt % of quaternary ammonium compound.

Embodiment 18 is the antimicrobial fibrous material of any of embodiments 1-4 or the method of any of embodiments 5-17, wherein the plurality of absorbent fibers includes cellulose fibers.

Embodiment 19 is the antimicrobial fibrous material of any of embodiments 1-4 and 18 or the method of any of embodiments 5-18, wherein the plurality of absorbent fibers absorbs at least 1000 wt % of water, based on the original dry weight of the fibers.

Embodiment 20 is the antimicrobial fibrous material of any of embodiments 1-4 and 18-19 or the method of any of embodiments 5-19, wherein the quaternary ammonium compound covalently grafted to the plurality of fibers includes at least one of monomers, oligomers and polymers covalently grafted to the plurality of fibers.

Embodiment 21 is the antimicrobial fibrous material of any of embodiments 1-4 and 18-20 or the method of any of embodiments 5-20, wherein the quaternary ammonium compound covalently grafted to the plurality of fibers is one of a plurality of quaternary ammonium compounds covalently grafted to the plurality of fibers, and wherein each of the quaternary ammonium compounds is a monomer, an oligomer or a polymer.

Embodiment 22 is the antimicrobial fibrous material of any of embodiments 1-4 and 18-21 or the method of any of embodiments 5-21, wherein at least some of the monomers include at least one of dimethyldecyl ammonium ethyl acrylate, dimethyldecyl ammonium ethyl methacrylate, dimethylhexadecyl ammonium ethyl acrylate, and dimethylhexadecyl ammonium ethyl methacrylate.

Embodiment 23 is the antimicrobial fibrous material of any of embodiments 1-4 and 18-22 or the method of any of embodiments 5-22, wherein the quaternary ammonium compound is covalently grafted to the plurality of fibers via covalent, non-siloxane bonds.

Embodiment 24 is the antimicrobial fibrous material of any of embodiments 1-4 and 18-23 or the method of any of embodiments 5-23, wherein the quaternary ammonium compound is covalently grafted to the plurality of fibers via carbon-carbon bonds.

Embodiment 25 is the antimicrobial fibrous material of any of embodiments 1-4 and 18-24 or the method of any of embodiments 5-24, wherein the plurality of absorbent fibers forms at least a portion of a woven, a non-woven, and a combination thereof.

The following working examples are intended to be illustrative of the present disclosure and not limiting.

### EXAMPLES

### Materials

Materials used for the examples are shown in Table 1.

TABLE 1
Materials List

| Material | Description | Source |
|---|---|---|
| DMAEA | Dimethylbutyl ammonium | CIBA, AGEFLEX |
| | ethylacrylate | FA 1 |
| DMAEMA | Dimethylhexadecyl | 3M Company, St. |
| | ammonium ethyl | Paul, MN |
| | methacrylate | |
| 1-Bromohexadecane | CAS # 112-82-3 | Sigma-Aldrich, St. |
| | | Louis, MO |
| 1-Bromobutane | CAS # 109-65-9 | Sigma-Aldrich, St. |
| | | Louis, MO |
| 1-Bromohexane | CAS # 111-25-1 | Sigma-Aldrich, St. |
| | | Louis, MO |
| 1-Bromodecane | CAS # 112-29-8 | Sigma-Aldrich, St. |
| | | Louis, MO |
| Sheep Blood | Defibrinated sheep | HemoStat |
| | blood | Laboratories, |
| | | Dixon, CA |
| BHI | Brain heart infusion | Becton, Dicksinson |
| | broth | and Company, |
| | | Franklin Lakes, NJ |
| BHT | 2,6-Di-tert-butyl-4- | Sigma-Aldrich, |
| | methylphenol, CAS # | St. Louis, MO |
| | 128-37-0 | |
| SR550 | Methoxy polyethylene | Sartomer Company, |
| | glycol (350) | Exton PA |
| | monomethacrylate | |
| Cellulose Fibers | Lyocell Tencel ®, 1.7 | Lenzing Fibers Inc., |
| | dtex, 38 mm | New York, NY |
| S. aureus | Staphylococcus aureus, | Microbiologies, |
| | ATCC# 25923 | Inc., St. Cloud, |
| | | MN |
| P. aeruginosa | Pseudomonas aeruginosa, | Microbiologies, |
| | ATCC# 9027 | Inc., St. Cloud, |
| | | MN |
| S. epidermidis | Staphylococcus epidermidis, | Microbiologies, |
| | ATCC# 14990 | Inc., St. Cloud, |
| | | MN |

### Test Methods

### Microbial Time Kill

Bacteria were grown overnight in tryptic soy broth at 37° C., then adjusted and diluted to ˜10⁶ concentration in phosphate buffer solution or sterile DI water. Approximately 0.25 g of fibers were placed into a sterile tube with 1 mL of bacteria inoculums. The fibers were left at room temperature for 30 min and 120 min. DE neutralizer was then added, and the tube vortexed for approximately 60 seconds. The number of surviving bacteria in the Neutralizing Broth was determined by using Petrifilm™ AC plates, (3M Company, St. Paul, Minn.). The samples were serially diluted and each dilution was plated onto the Petrifilm™. Plates were incubated for 48 hours at 37+/−1° C. After incubation, the colonies were counted and the number of surviving bacteria were calculated and are reported in Table 3. Log reduction was also calculated and is reported in Table 3. The mean of two tests was reported.

### Odor

Bacteria were grown overnight in tryptic soy broth at 37° C. One microliter of this overnight culture was added to a 30 mL BHI/20 mL defibrinated sheep blood mixture. Fibers were placed into a sterile 50 mL tube and inoculated with the diluted overnight culture. The open tubes were placed in anaerobic chamber to remove oxygen and sealed. The tubes were then incubated at 37° C. in an anaerobic chamber. Odor was evaluated after 5 and 7 days with a sniff test.

### Absorbency

Dry sample fibers were weighed to the nearest 0.0001 g, and the weight recorded as “A”. The fibers were then placed in a Petri dish and completely submerge with warmed deionized water. The Petri dish was placed in an oven set at 37° C. for 30 minutes ±5 minutes. After 30 minutes the Petri dish was removed from the oven and the sample was removed with tweezers and suspended for 30±2 seconds to allow for the excess water to drip off. The sample was reweighed to the nearest 0.0001 g and the weight recorded as “B”. Absorbency was calculated by the following equation:

$$Absorbency = \frac{B - A}{A} \times 100$$

### Examples

### Antimicrobial Preparation

### Synthesis of DMAEA-C₁₆Br Monomer

A three neck 3 L round bottom reaction flask equipped with overhead condenser, mechanical stirrer, and temperature probe was charged with 234 parts of DMAEA, 617 parts of acetone, 500 parts of 1-bromohexadecane, and 0.5 parts of BHT. The mixture was heated to 35° C. with stirring at 150 rpm. After 24 h of heating, the reaction mixture was cooled to room temperature. The clear reaction solution was transferred to a round bottom flask and acetone was removed by rotary evaporation under vacuum at 40° C. The resulting solid residue was mixed with 1 L cold ethyl acetate and mixed for 10 minutes. The mass was filtered and the solid product was washed with 500 mL cold ethyl acetate. The solids was transferred to a tray and dried overnight in vacuum oven at 40° C. This was the DMAEA-C₁₆Br monomer.

Additional antimicrobials (DMAEMA-C₁₆Br, DMAEA-C₄Br, DMAEA-C₆Br, DMAEA-C₁₀Br) were prepared in a similar fashion.

### Example 1 (E-1)

Cellulose fibers (6 grams) were placed in a plastic bag, purged with N₂, and sealed. The bag was then passed through an electron beam at a web speed of 19 feet per minute (fpm; 0.1 m/s). (Energy Sciences Inc., Electrocurtain CB-300, Wilmington, Mass.), delivering a dose of 5 MRad (50 kGy). After irradiation, the bag was placed in a purged glove box, fibers were removed from the bag and placed into a purged jar containing an aqueous solution of DMAEA-C₁₆ Br (1.2 g in 90 mL water). The sealed jar was left overnight at room temperature inside the purged glovebox. The jar was then opened and the polymerization reaction was quenched with atmospheric oxygen. The fibers were washed with water, methanol, isopropanol, and once again with water, followed by drying in an air-circulating oven at 40° C. for 24 h. The amount of antimicrobial monomer grafted onto the fibers was calculated from the difference between the initial weight of the fibers and the final weight of the fibers after grafting. Grafting yields were calculated by dividing the grams of monomer grafted onto the fibers by the total grams of monomer in the grafting solution. Grafting yields ranged from about 10% to 20%. Additional examples E-2-E-9 are shown in Table 2.

TABLE 2
Example Formulations

| | | | Amount in |
|---|---|---|---|
| Sample | Fiber (g) | Antimicrobial Monomer | Solution (g) |
| E-1 | 6 | DMAEA-C₁₆Br | 1.2 |
| E-2 | 6 | DMAEMA-C₁₆Br | 1.2 |
| E-3 | 6 | DMAEA-C₄Br | 1.2 |
| E-4 | 6 | DMAEA-C₆Br | 1.2 |
| E-5 | 6 | DMAEA-C₁₀Br | 1.2 |
| E-6 | 6 | DMAEA-C₁₆Br | 1.2 |
| E-7 | 6 | SR550/DMAEA-C₁₆Br ^{[a]} | 0.02/0.6 |
| E-8 | 6 | DMAEA-C₁₆Br | 1.4 |
| E-9 | 6 | DMAEMA-C₁₆Br | 1.4 |

^{[a]} Solution contained 0.02 g SR550 and 0.6 DMAEA-C₁₆Br

Results of microbial kill and odor are shown in Tables 3 and 4, respectfully.

TABLE 3
Microbial Kill Test Results

| | | | | | P. aeruginosa | |
|---|---|---|---|---|---|---|
| | S. aureus | | | | Log | Log |
| | Log CFU/cm² | | Log Reduction^{[a]} | | CFU/cm² | Reduction |
| Sample | 30 min | 120 min | 30 min | 120 min | 120 min | 120 min |
| Control^{[b]} | 7.0 | 6.3 | N/A | N/A | 6.4 | N/A |
| E-1 | 3.3 | 2.7 | 3.7 | 3.6 | Not | — |
| | | | | | tested | |
| E-2 | 5.8 | 4.5 | 1.2 | 1.8 | Not | — |
| | | | | | tested | |
| E-3 | Not | 5.9 | — | 0.4 | 6.2 | 0.2 |
| | tested | | | | | |
| E-4 | Not | 4.1 | — | 2.2 | 3.6 | 2.8 |
| | tested | | | | | |
| E-5 | Not | 1.0 | — | 5.3 | 1.0 | 5.4 |
| | tested | | | | | |
| E-6 | Not | 1.2 | — | 5.1 | 1.9 | 4.5 |
| | tested | | | | | |
| E-7 | Not | 1.0 | — | 5.3 | 1.0 | 5.4 |
| | tested | | | | | |

^{[a]}vs. Control
^{[b]}Fibers without grafted antimicrobial

TABLE 4
Odor Test Results

| Sample | | S. epidermidis | | P. aeruginosa | |
|---|---|---|---|---|---|
| | grams | Day 5 | Day 7 | Day 5 | Day 7 |
| Control^{[a]} | 100 | Malodor | Malodor | Malodor | Malodor |
| | 50 | Malodor | Malodor | Malodor | Malodor |
| | 25 | Malodor | Malodor | Malodor | Malodor |
| | 12 | Malodor | Malodor | Malodor | Malodor |
| E-8 | 100 | No odor | No odor | No odor | No odor |
| | 50 | No odor | No odor | No odor | No odor |
| | 25 | No odor | No odor | No odor | No odor |
| | 12 | No odor | No odor | Malodor | No odor |
| E-9 | 100 | No odor | No odor | No odor | No odor |
| | 50 | No odor | No odor | No odor | No odor |
| | 25 | No odor | Not tested | No odor | No odor |

^{[a]}Fibers without grafted antimicrobial

The absorbency of the cellulose fibers (i.e., unreacted, non-grafted starting material) used in the Examples was tested and compared to a polyester non-woven (Sontara™, DuPont, Wilmington, Del.). Results are shown in Table 5.

TABLE 5
Absorbency Data

| Sample | Absorbency (%) |
|---|---|
| Cellulose fibers | 2,410 |
| Polyester non-woven | 588 |

Various features and aspects of the present disclosure are set forth in the following claims.

1. An antimicrobial fibrous material for use in medical articles comprising:
a plurality of absorbent fibers; and
a quaternary ammonium compound covalently grafted to the plurality of fibers, wherein the quaternary ammonium compound is derived from quaternary amine-functionalized ethylenically unsaturated monomers, and wherein at least some of the monomers have the formula of Formula I: 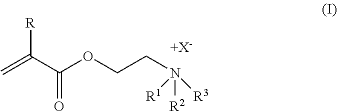
wherein:
R is selected from H and CH₃;
R¹ and R² are each selected from CH₃ and C₂H₅;
R³ is CₙH₂ₙ₊₁, where n ranges from 4 to 22; and
X is selected from Cl, Br, BF₄, N(SO₂CF₃)₂, O₃SCF₃, and O₃SC₄F₉.

2. The antimicrobial fibrous material of claim 1, wherein the antimicrobial fibrous material is free of catalyst.

3. The antimicrobial fibrous material of claim 1, wherein the antimicrobial fibrous material forms at least a portion of at least one of a wound dressing, an intravenous dressing, a wound or surgical packing material, a bodily orifice packing material, and a combination thereof.

4. The antimicrobial fibrous material of claim 1, wherein the antimicrobial fibrous material exhibits at least a 2 log reduction in gram positive or gram negative bacteria.

5. A method of making antimicrobial fibers for use in medical articles, the method comprising:
providing a plurality of absorbent fibers;
irradiating the plurality of fibers with high energy irradiation to generate a plurality of irradiated fibers;
providing quaternary amine-functionalized ethylenically unsaturated monomers, wherein at least some of the monomers have the formula of Formula I: 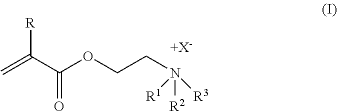
wherein:
R is selected from H and CH₃;
R¹ and R² are each selected from CH₃ and C₂H₅;
R³ is CₙH₂ₙ₊₁, where n ranges from 4 to 22; and
X is selected from Cl, Br, BF₄, N(SO₂CF₃)₂, O₃SCF₃, and O₃SC₄F₉; and
combining the plurality of irradiated fibers with the quaternary amine-functionalized ethylenically unsaturated monomers to form antimicrobial fibers with a quaternary ammonium compound covalently grafted thereto.

6. The method of claim 5, wherein combining the plurality of irradiated fibers with the quaternary amine-functionalized ethylenically unsaturated monomers occurs without the use of catalyst.

7. The method of claim 5, wherein the antimicrobial fibers are catalyst-free.

8. The method of claim 5, further comprising:
removing residual monomers from the antimicrobial fibers; and
drying the antimicrobial fibers after removing residual monomers from the antimicrobial fibers.

9. The method of claim 5, wherein providing quaternary amine-functionalized ethylenically unsaturated monomers includes providing an aqueous solution of quaternary amine-functionalized ethylenically unsaturated monomers, and wherein combining the irradiated fibers with the quaternary amine-functionalized ethylenically unsaturated monomers includes positioning the irradiated fibers in the aqueous solution of quaternary amine-functionalized ethylenically unsaturated monomers.

10. The method of claim 5, wherein the high energy irradiation includes at least one of electron beam irradiation and gamma irradiation.

11. The method of claim 5, wherein irradiating the plurality of fibers with a high energy irradiation and combining the plurality of irradiated fibers with the quaternary amine-functionalized ethylenically unsaturated monomers occur in an inert atmosphere.

12. The method of claim 5, wherein combining the plurality of irradiated fibers with the quaternary amine-functionalized ethylenically unsaturated monomers yields antimicrobial fibers comprising at least 2 wt % of quaternary ammonium compound.

13. The antimicrobial fibrous material of claim 1, wherein the plurality of absorbent fibers includes cellulose fibers.

14. The antimicrobial fibrous material of claim 1, wherein the plurality of absorbent fibers absorbs at least 1000 wt % of water, based on the original dry weight of the fibers.

15. The antimicrobial fibrous material of claim 1, wherein the quaternary ammonium compound covalently grafted to the plurality of fibers includes at least one of monomers, oligomers and polymers covalently grafted to the plurality of fibers.

16. The antimicrobial fibrous material of claim 1, wherein at least some of the monomers include at least one of dimethyldecyl ammonium ethyl acrylate, dimethyldecyl ammonium ethyl methacrylate, dimethylhexadecyl ammonium ethyl acrylate, and dimethylhexadecyl ammonium ethyl methacrylate.

17. The antimicrobial fibrous material of claim 1, wherein the quaternary ammonium compound is covalently grafted to the plurality of fibers via covalent, non-siloxane bonds.

18. The antimicrobial fibrous material claim 1, wherein the quaternary ammonium compound is covalently grafted to the plurality of fibers via carbon-carbon bonds.

19. The antimicrobial fibrous material of claim 1, wherein the plurality of absorbent fibers forms at least a portion of a woven, a non-woven, and a combination thereof.
